(19) United States
(12) Patent Application Publication
(10) Pub. No.: US 2026/0306135 A1
(43) Pub. Date: Oct. 8, 2026

(54) NEEDLE FOR DELIVERY OF MULTIPLE EXTENDING ELEMENTS TO TISSUE

(71) Applicant: University of Pittsburgh – Of the Commonwealth System of Higher Education, Pittsburgh, PA (US)

(72) Inventors: Gaurav Chauhan, Indianapolis, IN (US); Trent D. Emerick, Mars, PA (US)

(21) Appl. No.: 19/473,524

(22) PCT Filed: May 10, 2024
(86) PCT No.: PCT/US2024/028796

§ 371 Date: Oct. 8, 2025

Related U.S. Application Data
(60) Provisional application No. 63/465,902, filed on May 12, 2023.

(51) Int. Cl. A61B 17/34; A61B 17/00; A61B 90/00; A61N 1/372

(52) U.S. Cl. CPC A61B 17/3468; A61B 17/3401; A61B 17/3417; A61B 90/03; A61N 1/372; A61B 2017/00477; A61B 2090/034

(57) ABSTRACT

A needle system for implanting an extending element including a needle body—configured to be placed in closed state and in open state, wherein an axially extending opening is provided in the needle body in the open state, whereby the extending element, which has been passed through a lumen of the needle body, can be removed from the lumen via the axially extending opening.

### CROSS-REFERENCE TO RELATED APPLICATIONS

This application claims benefit of U.S. Provisional Patent Application Ser. No. 63/465,902, filed May 12, 2023, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

The placement of spinal cord stimulator leads for use in chronic pain relief is associated with considerable risks, including, for example, dural puncture, infection, bleeding, and nerve injury. Approximately 100,000 spinal cord stimulators are placed in the United States each year, and roughly the same amount are placed in the European Union.

Numerous studies have shown the benefits of spinal cord stimulation for various types of chronic pain. The stimulator leads used in spinal cord stimulation are placed along the spinal cord in the epidural space through a hollow needle that is precisely positioned under x-ray guidance. Hollow bore needles (also known as a Tuohy needle) are used to deliver the stimulator lead into the epidural space as well as to other regions of interest. Chronic pain specialists use hollow epidural needles (typically, 14-gauge epidural needles) to correctly place spinal cord stimulator leads into the epidural space. The needles have a cutting surface (bevel) to accurately advance through the various tissue layers, which is helpful. However, that sharp surface can also be inadvertently advanced too far. If the needle extends beyond the epidural space into the dural sac, the patient is at risk of numerous complications, including dural puncture headache, cerebrospinal fluid leak, infection, neural injury, or paraplegia. Typically, it is standard practice to place at least two spinal cord stimulator leads side-by-side in the epidural space to maximize the surface area of electrical stimulation of the spinal cord. That practice requires the hollow needle to be inserted twice, because each hollow needle has the capacity to place only one stimulator wire therethrough. Each such insertion and placement may take up to 30 minutes. Requiring two placements doubles the risk of complications as a new needle is inserted immediately adjacent to the first needle once the first stimulator wire is placed. A second needle placement is also associated with additional post-procedural pain arising from the extra puncture or incision site, higher risk of infection, and risks associated with longer procedure/anesthesia times.

### SUMMARY

In one aspect, a needle system for implanting an extending element includes a needle body configured to be placed in a closed state and in an open state. An axially extending opening is provided in the needle body in the open state, whereby the extending element, which has been passed through a lumen of the needle body, can be removed from the lumen via the axially extending opening.

In a number of embodiments, the needle body includes a first body section and a second body section. The second body section is movable relative to the first body section to place the axially extending opening formed in the first body section in the open state (as well as in the closed state). In the open state of the axially extending opening/needle body, the extending element, which has been passed through a lumen of the needle body, can be removed from the lumen via the axially extending opening as described above.

In a number of embodiments, the second body section is axially slidable relative to the first body section to remove the second body section from connection with the first body section to place the axially extending opening in the open state (and to be placed in connection with the first body section to place the axially extending opening in the closed state) . One of the first body section and the second body section may, for example, include laterally extending flanges and the other of the first body section and the second body section may include grooves positioned to seat the laterally extending flanges. In a number of other embodiments, the second body section is rotatable relative to the first body section to place the axially extending opening in an open state or a closed state.

The needle system may further include a cap removably connectible to a proximal end of the needle body. In a number of embodiments, the cap includes a passage therethrough which connects with the lumen when the cap is connected to the proximal end of the needle body. In a number of embodiments wherein the second body section is axially slidable relative to the first body section to remove the second body section from connection with the first body section when the cap is disconnected from the needle body, the cap secures the second body section in connection with the first body section when the cap is connected to the needle body. In a number of embodiments, a first connector section is formed on a proximal end of the first body section and a second connector section is formed on a proximal end of the second body section. The first connector section and the second connector section form a connector when the first body section and the second body section are connected such that the axially extending opening is in a closed state. The cap includes a cooperating connector to form a removable connection with the connector on a distal end thereof. The cap may further include a connector (for example, a Luer connector) on a proximal end thereof for connection of, for example, a syringe thereto.

In a number of embodiments, the needle system further includes a stylet (or plunger) configured to be passed through the lumen of the needle body when the needle body is in the open stated (for example, when the second body section is moved relative to first body section to place the axially extending opening in the open state). Passing the stylet through the lumen may be used to displace the extending element to a position from the lumen to a position outside of the needle body.

In a number of embodiments, the needle system further includes one or more movable elements positioned within the lumen of the needle body. The one or more movable elements are movable to abut and displace the extending element from the lumen to a position outside of the needle body (when the needle body is placed in the open state).

In another aspect, a method of implanting one or more extending elements includes inserting at least a portion of a needle body of a needle system into tissue of a patient while the needle body is in a closed state, passing the extending element from external to the patient into a lumen of the needle body to extend to a region of interest, opening an axially extending opening in the needle body to place the needle body in an open state, and moving a portion of the extending element passing through the lumen from the lumen to outside of the needle body through the axially extending opening.

In a number of embodiments, the needle body includes a first body section and a second body section, and the method includes moving the second body section relative to the first body section to place an axially extending opening formed in the first body section in an open state. Moving the second body section relative to the first body section may, for example, include axially moving/sliding the second body section relative to the first body section to remove the second body section from connection with the first body section. In a number of embodiments, one of the first body section and the second body section includes laterally extending flanges and the other of the first body section and the second body section includes grooves positioned to seat the laterally extending flanges. Moving the second body section relative to the first body section may alternatively include rotating the second body section relative to the first body section.

In a number of embodiments, a cap is removably connectible to a proximal end of the needle body. The cap may, for example, include a passage therethrough which connects with (or is placed in fluid connection with) the lumen when the cap is connected to the proximal end of the needle body. In a number of embodiments (for example, in which the second body section is axially slidable relative to the first body section to remove the second body section from connection with the first body section when the cap is disconnected from the needle body) the cap secures the second body section in connection with the first body section when the cap is connected to the needle body.

In a number of embodiments, a first connector section is formed on a proximal end of the first body section and a second connector section is formed on a proximal end of the second body section. The first connector section and the second connector section form a connector when the first body section and the second body section are connected and the axially extending opening is in a closed state. The cap may, for example, include a cooperating connector to form a removable connection with the connector.

In a number of embodiments, the method further includes passing a stylet (or plunger) through the lumen of the needle body when the second body section is moved relative to first body section to place the axially extending opening in an open state to move the extending element from the lumen via the axially extending opening to a position outside of the needle body. In a number of embodiments, the method further includes actuating one or more movable elements positioned within the lumen of the needle body. The movable elements are movable to abut and move the extending element from the lumen to a position outside of the needle body when the axially extending opening is in an open state.

At least a second extending element may be passed from external to the patient into the lumen of the needle body to extend to the region of interest or a second region of interest after the extending element is moved from the lumen to a position outside of the needle body. The method may, for example, further include moving the second body section relative to first body section while the portion of the needle body remains inserted into tissue of the patient to place the axially extending opening formed in the first body section in a closed state after moving the extending element from the lumen through the axially extending opening, passing a second extending element from external to the patient into the lumen of the needle body to extend to the region of interest or a second region of interest, moving the second body section relative to first body section to place the axially extending opening in an open state; and moving a portion of the second extending element passing through the lumen from the lumen to outside of the needle body through the axially extending opening.

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates an isometric view of an embodiment of a needle hereof for delivery of extending elements such as catheters and wires/leads wherein the first or main body section and the second or secondary body section are in a connected or closed state.

FIG. 1B illustrates a side view of the needle of FIG. 1A.

FIG. 1C illustrates a cross-sectional view of the needle of FIG. 1A along section A-A (see FIG. 1B).

FIG. 1D illustrates a top view of the needle of FIG. 1A.

FIG. 1E illustrates a side, cross-sectional view along section B-B (see FIG. 1D).

FIG. 2A illustrates an exploded, isometric view of the needle of FIG. 1A wherein the first or main body section and the second or secondary section are in a disconnected or open state.

FIG. 2B illustrates an exploded, isometric view of the needle of FIG. 1A.

FIG. 2C illustrates an exploded side view of the needle of FIG. 1A.

FIG. 3A illustrates an isometric view of the body or main body of the needle of FIG. 1A.

FIG. 3B illustrates an isometric view of the body of the needle of FIG. 1A.

FIG. 3C illustrates a top view of the body of the needle of FIG. 1A.

FIG. 3D illustrates a top view of the body of the needle of FIG. 1A with an embodiment of a stylet or plunger within the bore thereof to expel an extending member such as a wire/lead from the bore thereof.

FIG. 3E illustrates a front or distal view of the body of the needle of FIG. 1A.

FIG. 3F illustrates a side view of the body of the needle of FIG. 1A.

FIG. 3G illustrates a cross-sectional view of the body of the needle of FIG. 1A along section C-C (see FIG. 3F).

FIG. 4A illustrates an isometric view of a removable section of the body of the needle of FIG. 1A.

FIG. 4B illustrates another isometric view of a removable, second section of the body of the needle of FIG. 1A.

FIG. 4C illustrates a rear view of a removable, second section of the body of the needle of FIG. 1A.

FIG. 4D illustrates a top view of a removable, second section of the body of the needle of FIG. 1A.

FIG. 4E illustrates a side view of a removable, second section of the body of the needle of FIG. 1A.

FIG. 4F illustrates a cross-sectional view of a removable, second section of the body of the needle of FIG. 1A along section D-D (see FIG. 4E).

FIG. 5A illustrates a front view of an embodiment of a cap or closure of the needle of FIG. 1A.

FIG. 5B illustrates an isometric view of the cap or closure of the needle of FIG. 1A.

FIG. 5C illustrates a side view of the cap or closure of the needle of FIG. 1A.

FIG. 5D illustrates a cross-sectional view of the cap or closure of the needle of FIG. 1A along section E-E (see FIG. 5C).

FIG. 6A illustrates a side view of an embodiment of a stylet or plunger hereof.

FIG. 6B illustrates an isometric view of the stylet or plunger of FIG. 6A.

FIG. 7 illustrates an embodiment of a plurality of actions taken to deliver multiple extending elements such as wires/leads using the needle of FIG. 1A, wherein the needle is illustrated in top views.

FIG. 8A illustrates a cutaway axial view of another embodiment of a needle body of a needle or needle system hereof wherein the first, outer or main body section and the second, inner or secondary body section of the needle body are slidably positioned relative to each other to place the needle body in a closed state.

FIG. 8B illustrates a cutaway axial view of the needle body of FIG. 8A wherein the second body section is slid relative to the first body section along the axis of the needle body to remove the second body section from within the first body section to place the needle body in an open state.

FIG. 8C illustrates a side view of the needle body of FIG. 8A wherein the second body section has been slid in the axial direction relative to the first body section to be removed from the first body section and place the needle body in the open state.

FIG. 9A illustrates a cutaway axial view of another embodiment of a needle body of a needle or needle system hereof wherein the first, outer or main body section and the second, inner or secondary body section of the needle body are rotatably positioned relative to each other to place the needle body in a closed state.

FIG. 9B illustrates a cutaway axial view of the needle body of FIG. 9A wherein the second body section is rotated relative to the first body section about the axis of the needle body to place the needle body in an open state.

FIG. 9B illustrates an enlarged cutaway axial view of the second body section of the needle body of FIG. 9A.

FIG. 10A illustrates a cutaway axial view of another embodiment of a needle body of a needle or needle system hereof wherein the first body section and the second body section of the needle body are rotatably positioned relative to each other to place the needle body in a closed state.

FIG. 10B illustrates a cutaway axial view of the needle body of FIG. 10A wherein the second body section is rotated relative to the first body section about the axis of the needle body to place the needle in an open state, and an extending member, which is positioned within the lumen of the needle body, abuts/applies force to a movable element within the lumen of the needle to move/displace the extending element from the lumen.

FIG. 10C illustrates a cutaway axial view of the needle of FIG. 10A wherein the second body section is rotated relative to the first body section to partially return the needle body to a closed state.

FIG. 11A illustrates a cutaway axial view of another embodiment of a needle body of a needle or needle system hereof wherein the first body section and the second body section of the needle body are rotated relative to each other to place the needle body in a closed state.

FIG. 11B illustrates a cutaway axial view of the needle body of FIG. 11A wherein the second body section is rotated relative to the first body section about the axis of the needle body to place the needle in an open state, and one or more movable/expandable elements such as a balloon or bladder, which is/are positioned within the lumen of the needle body, is/are expanded to abut/apply force to an extending element within the lumen of the needle body to move/displace the extending element from the lumen.

FIG. 12A illustrates a top view of another embodiment of a needle or needle system hereof wherein the needle body is in a closed state and is scored or otherwise weekend to allow opening thereof to an open state after insertion.

FIG. 12B illustrates a cross-sectional view of the needle body of FIG. 12A along section F-F in a closed state.

FIG. 12C illustrates a cross-section view of the needle body of FIG. 12A after application of a generally radially outward force thereto to place the needle body in an open state.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a needle” includes a plurality of such needles and equivalents thereof known to those skilled in the art, and so forth, and reference to “the needle” is a reference to one or more such needles and equivalents thereof known to those skilled in the art, and so forth. Recitation of ranges of values herein are merely intended to serve as a shorthand method of referring individually to each separate value falling within the range. Unless otherwise indicated herein, each separate value, as well as intermediate ranges, are incorporated into the specification as if individually recited herein. All methods described herein can be performed in any suitable order unless otherwise indicated herein or otherwise clearly contraindicated by the text.

As described above, hollow bore needles may be used to deliver/place an extending element such as a catheter or a stimulator lead to extend through tissue to a region of interest. For example, epidural needles such as Tuohy needles may be used to place epidural catheters in pregnant females. Further, chronic pain specialists may, for example, use epidural needles (for example, 14G epidural needles) to place stimulator leads (for example, in the epidural space, along the spinal cord). Because only a single lead can be passed through the lumen of a needle, under current practices, multiple needles (and multiple transcutaneous needle punctures) are required to place more than one lead. The internal diameter of the hollow needle cannot be easily enlarged to place two needles through the same lumen as this would require a large skin incision for placement.

Needles, needle systems, and methods hereof significantly reduce the risk of complications (for example, by 50% in the case of placement of two leads and by even a greater percentage in the case of placement of greater than two leads). Moreover, the procedure time is cut approximately in half for placement of two stimulator leads (and by even a greater percentage in the case of placement of greater than two leads). Needles, needle systems, and methods hereof thus significantly improve the safety profile and risk profile of patients receiving this procedure.

In a number of embodiments of needles or needle systems hereof, a hollow bore needle includes a section of a side wall thereof which is configured or operative to be placed in an open state after penetration of tissue so that an extending element (for example, a catheter or a wire/lead) passing through the lumen can be removed from the lumen of the needle (and positioned in the surrounding tissue) via the opening in the side wall of the needle body without removal of the needle from the tissue. A second extending element can subsequently be inserted into the needle lumen and the process repeated to position the second extending element. Two or more extending elements can be inserted into the tissue in the above-described manner. The needle may be repositioned within the tissue (to, for example, appropriately position subsequently inserted extending elements/leads) between insertion of the extending elements without removing the needle from the tissue.

Needles or needle systems hereof may, for example, be used to thread a spinal cord stimulator lead to the epidural space. Subsequently, the same needle can be used to place a second (third, etc.) lead, without removing the needle from the insertion site in the back. The needles, needle systems, and methodologies hereof thus eliminate the requirement of insertion of a second (third, etc.) epidural needle and mitigate/eliminate additional trauma associated therewith. As described above, risks of inadvertent dural puncture, cerebrospinal fluid leak, dural puncture headache, and nerve injury causing sensory and/or motor changes are minimized with fewer needle passes and needle placements. Procedural time is also reduced. The needles and needle systems may also be used as a teaching tool. Although representative embodiments of needles hereof are discussed in connection with placement of stimulator leads in the epidural space, the needles hereof may be used to place extending elements elsewhere (for example, near peripheral nerves, in brain tissue, for vascular access etc.).

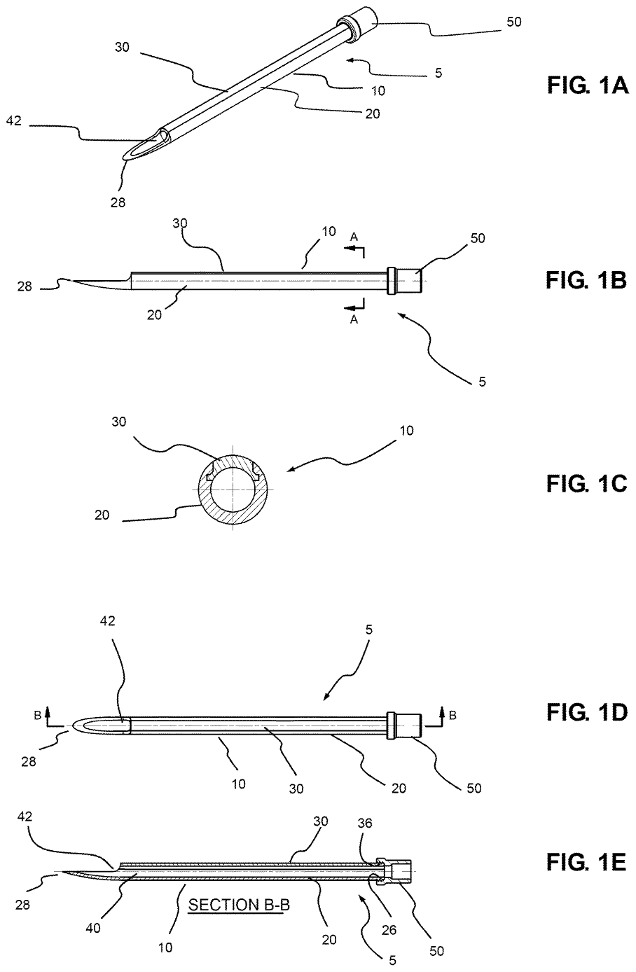

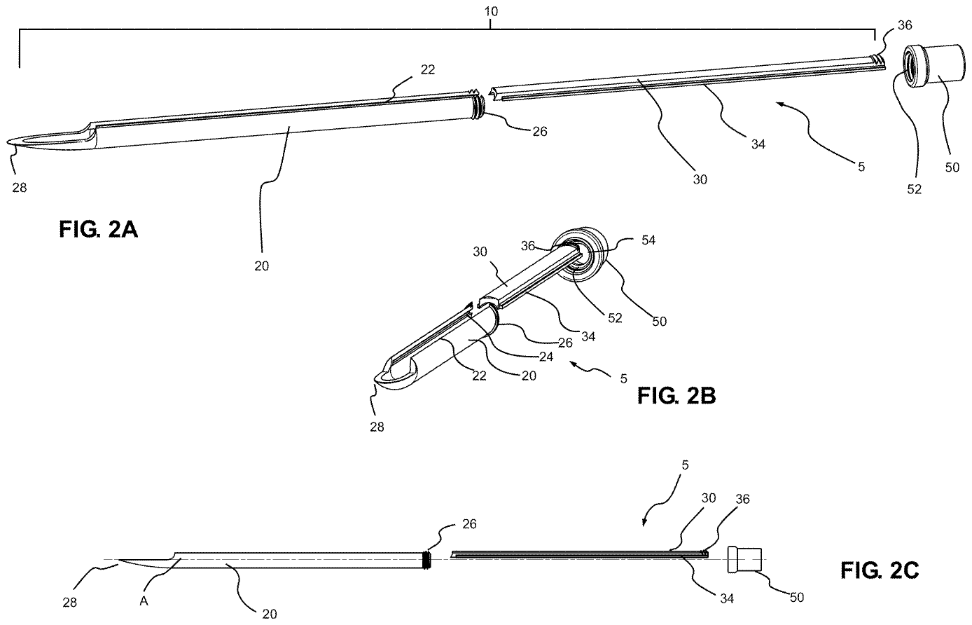

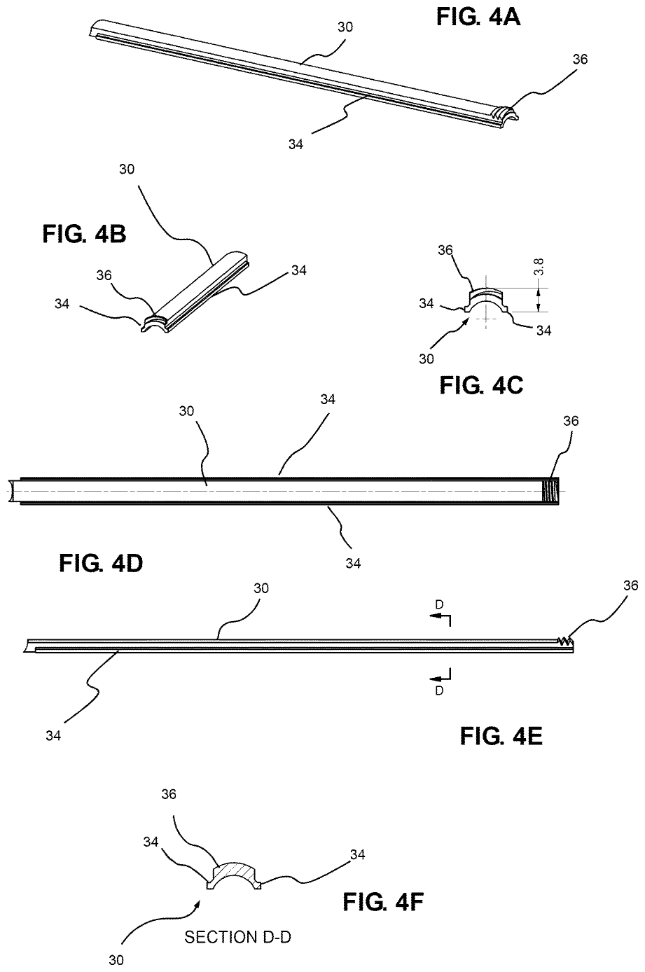

FIGS. 1A through 2C illustrates a representative embodiment of a needle system 5 hereof. Needle system 5 includes an extending needle body 10 which is the tissue penetrative portion of needle system 5. In the illustrated embodiment, needle body 10 is similar to a Tuohy epidural needle (and may include lumen diameter similar to currently used Tuohy needles), but includes a first or main body section 20 (see, for example, FIGS. 1A through 3F) and a second or secondary body section 30 (sometimes referred to as a slat) which is movable or removable relative to main body section 20 to expose an axially extending opening 22 (see, for example, FIGS. 2A and 2B) formed in main body section 20. In the illustrated embodiment, secondary body section 30 (see FIGS. 1A through 2C and 4A through 4F) includes laterally extending flanges 34 on each lateral side thereof which cooperate with seatings or grooves 24 formed in main body section 20 (see, for example, FIG. 2B). Flanges 34 slide relative to main body section 20 in an axial direction (defined by axis A of needle system 5 as illustrated in, for example, FIG. 2C) within grooves 24 to place opening 22 in an open or closed state (compare FIG. 1A and FIG. 2A, for example). In FIGS. 2A through 2C, secondary body section 30 is aligned to be connected to main body section 20 as described above.

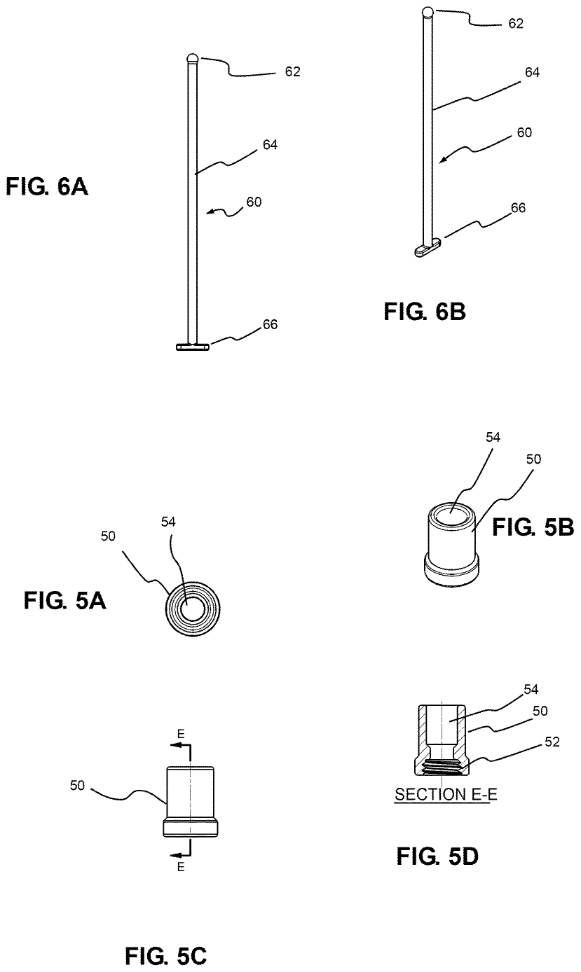

When secondary body section 30 is slid relative to main body section 20 to a fully connected or closed position as illustrated in FIGS. 1A through 1E, an enclosed, needle lumen or bore 40 of extending needle body 10 is formed. A forward or distal section of extending needle body 10 includes an opening 42 near a distal terminus of lumen 40. A proximal or rearward end of each of main body section 20 and secondary body section 30 includes a portion or section of a connector such as portion or section of threading 26 and 36, respectively. A forward or distal end of main body section 20 include a piercing/cutting edge 28. When secondary body section 30 is slid relative to main body section 20 to a fully connected or closed position as illustrated in FIGS. 1A through 1E, a connector (for example, threading) is formed from sections 26 and 36 to connect to a cooperating connector 52 (for example, cooperating threading) of a cap, coupler, or needle port 50 (see, for example, FIGS. 1E and 5A through 5D). In a number of embodiments, a distal end cap 50 includes a Luer or Luer-lock connector/coupler as known in the medical arts. A syringe may, for example, be placed in fluid connection with the distal end of cap 50 via such a Luer connector to, for example, deliver saline in a loss of resistance (LOR) technique as described further below.

In that regard, the epidural space is a thin, adipose-filled structure that lines the dural sac (dura). Outside (superficial) to the posterior epidural space is the ligamentum flavum. A loss of resistance or LOR technique is traditionally used to access the epidural space for stimulator placement, or injections of medications through a hollow needle. In an LOR technique, a needle is advanced (with or without x ray guidance) to close proximity to the ligamentum flavum. A specialized LOR syringe of saline and/or air with a plunger is then attached to the Luer-lock at the end of the needle, and a small amount of pressure is placed on the specialized LOR syringe plunger. The needle is then advanced in very small (1 millimeter) increments. When the needle bevel is inside the ligamentum flavum, there will be significant resistance to injection of the saline or air. When the needle bevel is advanced beyond the ligamentum and into the epidural space, the plunger will glide down when pushed with a small amount of pressure, indicating the needle bevel is in the epidural space. Contrast dye or x ray may also be used to determine final needle position once in the epidural space.

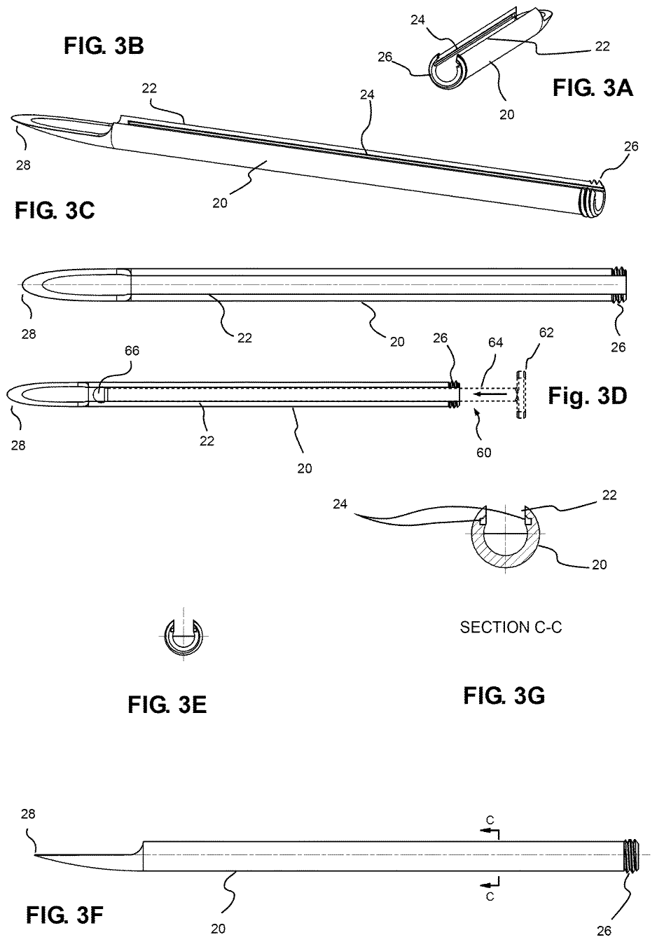

As illustrated in, for example, FIGS. 3D and 6A through 6B, needle system 5 further includes a stylet or plunger 60 which is used to force an extending element such as a wire/lead from lumen 40 when needle body 10 is in an open state. In the illustrated, representative embodiment, stylet 60 includes a forward or distal end section 62 that has a diameter similar to the diameter of lumen 40. End section 62 is connected to an extending member 64 at a forward or distal end of extending member 64. A handle 66 may optionally be connected to the rearward or proximal end of extending member 64.

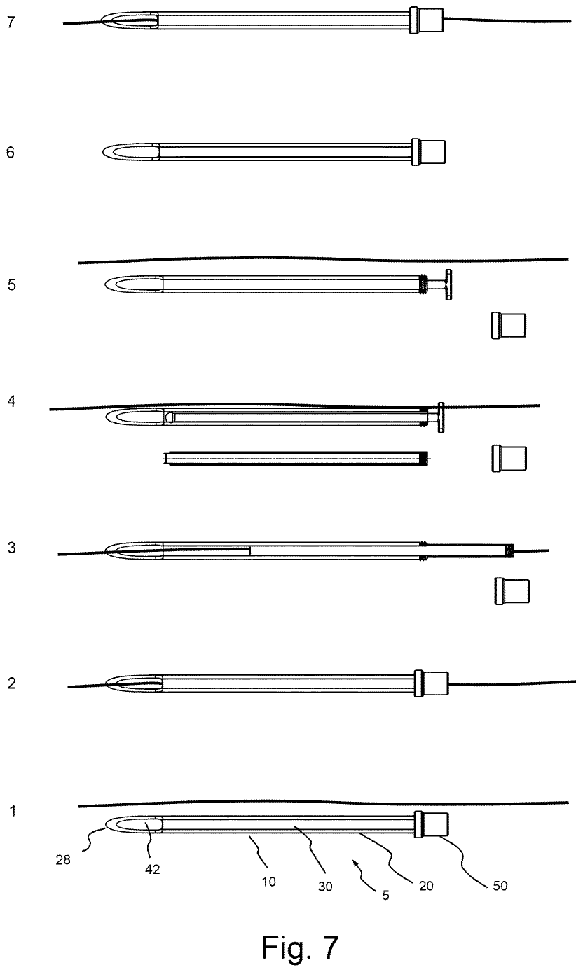

The operation of needle system 5 to place multiple extending elements within tissue can be described with reference to FIGS. 1A through 7, and particularly with reference to panels 1 through 7 of FIG. 7. As illustrated in panels 1 and 2, after penetration/implantation of a position of needle body 10 into the patient, an extending element such as a stimulator wire/lead 100 may passed through an opening 54 in cap 50, through lumen 40 (which is in connection with opening 44), and exit opening 42, to be positioned within a region of interest such as an epidural space. Subsequently, cap 50 is removed and secondary body section 30 is removed by sliding it rearward relative to main body section 20 as illustrated in panel 2 of FIG. 7. As illustrated in panels 4 and 5, stylet 60 is then inserted into lumen 40 (which is in fluid connection with the surrounding tissue via axially extending opening 42) to push wire/lead 100 into the surrounding tissue with the distal end of wire/lead 100 positioned within a predetermined volume of epidural space. As discussed further below, in other embodiments, a movable element (for example, an inflatable element, a biased element, or other element) may be positioned within lumen 40 so that use of stylet 60 is not required to displace extending element 100 from lumen 40.

As illustrated in panel 6, secondary body section 30 may then be replaced by engaging it with main body section 20 (as described above) and sliding it forward relative to main body section 20. Once needle body 10 is in a closed state (as, for example, illustrated in FIGS. 1A through 1E) cap 50 is attached. As illustrated in panel 7, the process described above may then be repeated upon insertion of another wire/lead 100 as described above. The process may be repeated to position 2 or more extending elements within a region of interest such as the epidural space. As described above, the forward or distal end of needle body may be repositioned within the tissue between insertion of the extending elements without removing the needle from the tissue to place extending elements at different positions.

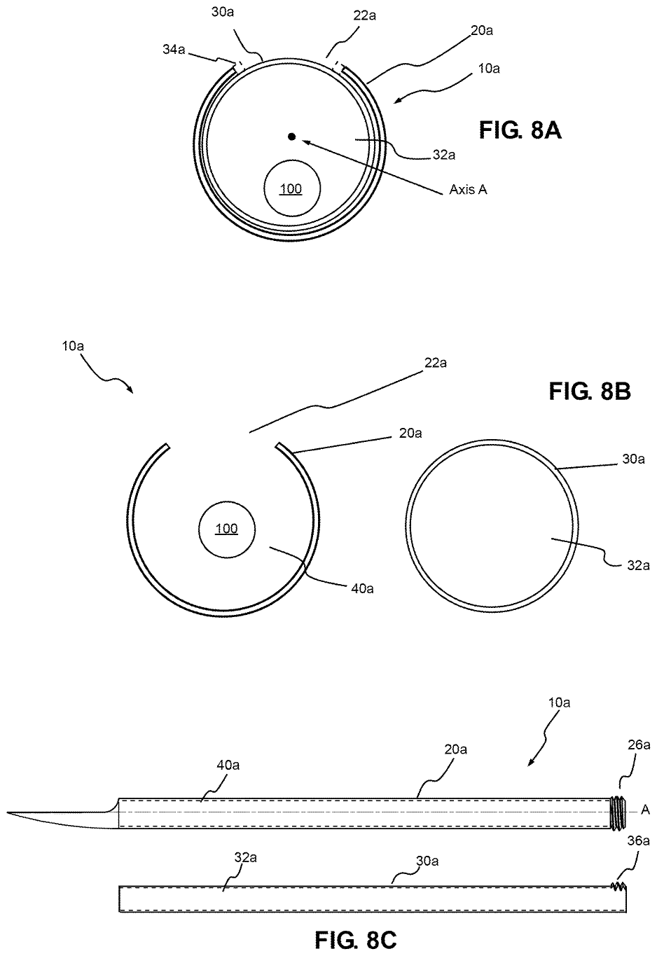

FIGS. 8A through 8C illustrates a cutaway axial view of another embodiment of a needle body 10a of a needle or needle system 5 hereof. In the illustrated embodiment of FIGS. 8A through 8C, a first, outer or main body section 20a and a second, inner or secondary body section 30a are slidably positioned relative to each other to place the needle 5 in a closed state (FIG. 8A) and in an open state (FIG. 8B). In the illustrated embodiment, second body section 30a is formed as a generally cylindrical element. An axially extending opening 22a, similar to opening 22 described above, is formed in first body section 20a. In the embodiment of FIGS. 8A through 8C, the open/closed state of opening 122a and needle body 10a is controlled by sliding of second body section 30a axially relative to first body section 20a in direction of the axis of needle body 10a similar to the disengagement/engagement of second body section 30 from first body section 20. Referring to FIG. 8A, extending element 100 may be inserted into a lumen 32a of second body section 30a. In the open state, after removal of second body section 30a from operative connection with first body section 20a as illustrated in FIGS. 8B and 8C, a stylet or plunger such as stylet 60 can be used to apply a displacing force to extending element 100 passing through lumen 40a to move/displace extending element 100 from the lumen. As illustrated schematically, an engagement mechanism 34a (for example, a cooperating flange/seating system,) can be provided to form an engagement (which may be a sealing engagement) between first body section 20a and second body section 30a. A proximal or rearward end of each of main body section 20a and secondary body section 30a includes a portion or section of a connector such as portion or section of threading 26a and 36a, respectively, for connection to a cap as described in connection with needle system 5.

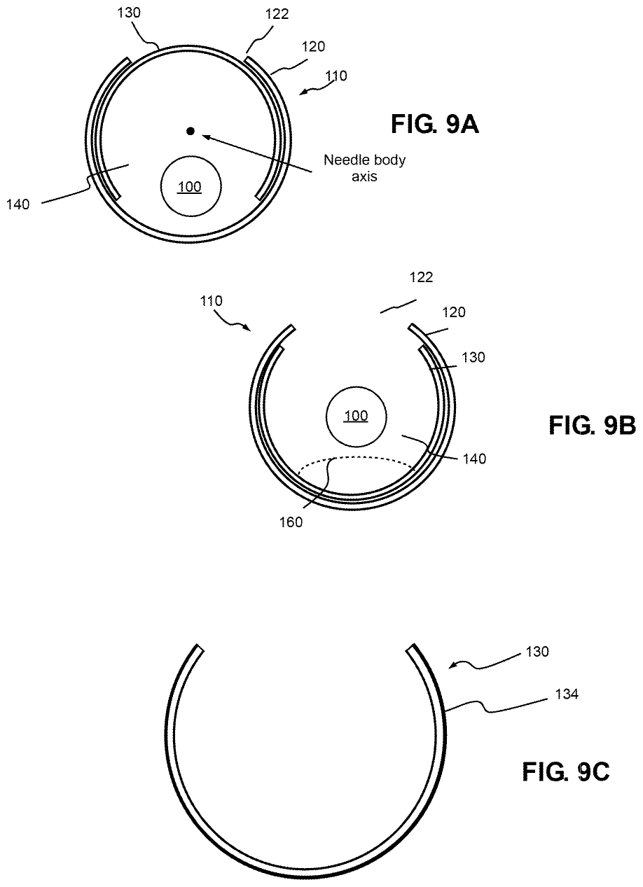

FIGS. 9A through 9C illustrate a cutaway axial view of another embodiment of a needle body 110 of a needle or needle system hereof. In the illustrated embodiment, a first, outer or main body section 120 and a second, inner or secondary body section 130 are rotatably positioned relative to each other to place the needle in a closed state. In that regard, an axially extending opening 122, similar to opening 22 described above, is formed in first body section 120. In the embodiment of FIGS. 9A through 9C, the open/closed state of opening 122 and needle body 110 is controlled by rotation of second body section 130 relative to first body section 120 about the axis of needle body 110 as illustrated in a comparison of FIGS. 9A and 9B. In the open state, as illustrated in FIG. 9B, a stylet or plunger such as stylet 60 can be used to apply a displacing force to extending element 100 passing through the lumen to move/displace extending element 100 from the lumen. Alternatively, one or more movable elements (for example, inflatable elements such as one or more bladders or balloons) can be positioned within lumen 140 (for example, connected to an inner wall of second body section 130) to move/displace extending element 100 from lumen 140 and into surrounding tissue upon actuation thereof. As illustrated for example in the enlarged view of second body section 130 illustrated in FIG. 9C, a sealing mechanism such as sealing mechanism/layer 134 (for example, a layer of relatively low friction, elastomeric or other sealing polymeric material material) can be provided on an outer surface of second body section 130 (or otherwise between first body section 120 and second body section 130) to form a sealing engagement between first body section 120 and second body section 130 to, for example, facilitate an LOR procedure as described above.

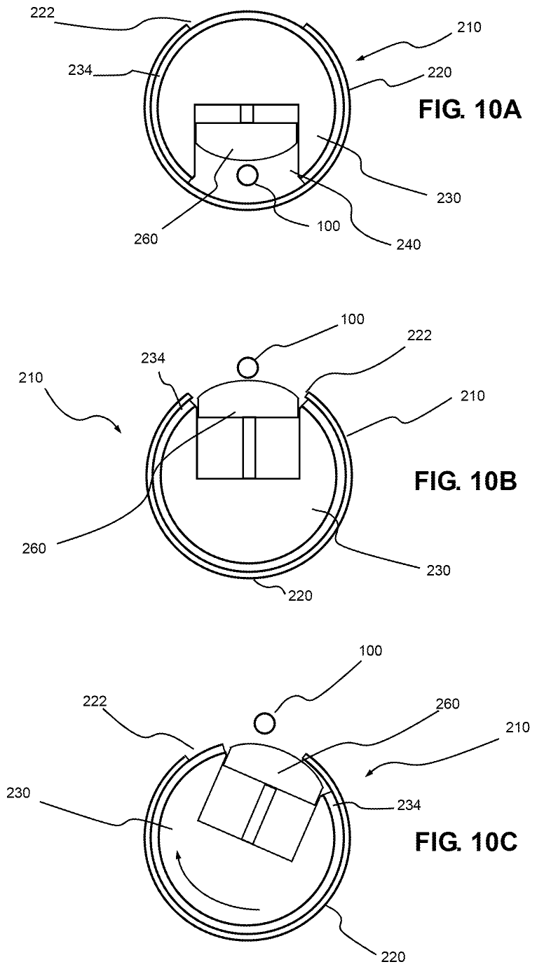

FIG. 10A illustrates a cutaway axial view of another embodiment of a needle body 210 of a needle or needle system hereof wherein the first body section 220 and the second body section 230 of needle body 210 are rotatably positioned relative to each other to place needle body 210 in a closed state. As illustrated in a comparison of FIGS. 10A and 10B, second body section 230 may be rotated about the axis of needle body 210 relative to first body section 220 to place needle body 210 in an open/closed state. In the open state, extending element 100 may be displaced from lumen 240 to a volume/tissue surrounding or adjacent needle body 210 via axially extending opening 222. In the illustrated embodiment, one or more movable elements 260 (for example, one or more biased movable elements) positioned within lumen 240 applies force to extending element 100 positioned within the lumen of the needle to move/displace extending element 100 from the lumen into the surrounding tissue (outside of needle body 210). Such movable elements may, for example, be extended (see FIG. 10B) and retracted radially (see FIGS. 10A and 10C) within lumen 240 as a function of the rotational position of second body section 230 (around the axis of needle body 210) relative to first body section 220 (with or without user intervention). FIG. 10C illustrates a cutaway axial view of the needle body 210 wherein second body section 230 is rotated relative to first body section 220 to partially return the needle body 210 to a closed state and wherein movable element is in a partially retracted state. A sealing mechanism 234 (for example, a layer of relatively low friction, elastomeric or other sealing polymeric material material) can be provided on an outer surface of second body section 230 to form a sealing engagement between first body section 220 and second body section 230.

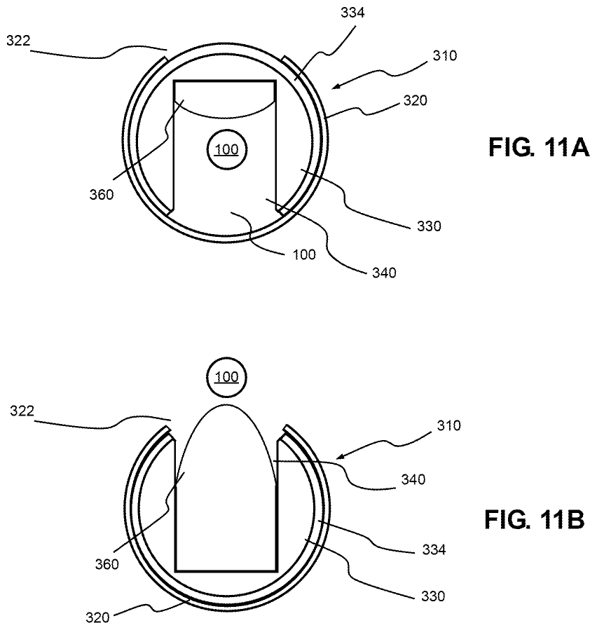

FIG. 11A illustrates a cutaway axial view of another embodiment of a needle body 310 of a needle or needle system hereof wherein a second body section 330 is rotatable about the axis of needle body 310 relative to first body section 320 to control an open/closed state of needle body 310 (compare FIGS. 11A and 11B). One or more movable elements in the form of one or more expandable balloons or bladders 360 is/are positioned within lumen 340 of needle body 310 to abut/apply force to and thereby move/displace extending element 100 from the lumen into the surrounding tissue. A sealing mechanism 234 (for example, a layer of relatively low friction, elastomeric or other sealing polymeric material material) can be provided on an outer surface of second body section 330 to form a sealing engagement between first body section 320 and second body section 330.

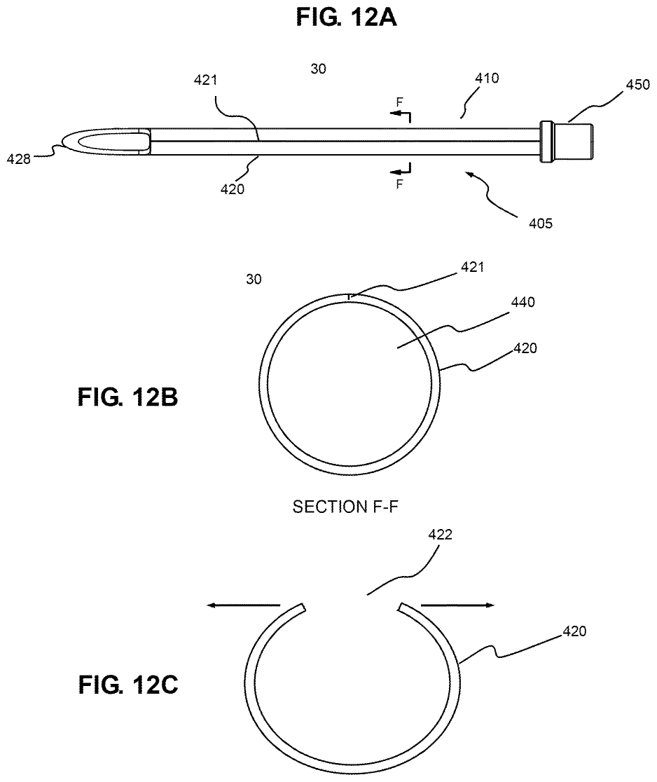

FIGS. 12A through 12C illustrated an embodiment of a needle or needle system 405 in which a needle body section 420 of a needle body 410 include a “scored” or otherwise weakened portion or section 421 which extends axially along a length thereof (formed along an axial line in the illustrated embodiment). Needle body section 420 may, for example, be scored to nearly the breaking or separation point as, for example, illustrated in FIG. 12B. After access of the epidural or other internal space, a stylet (not shown) with a sufficiently large (in diameter) distal end element may be inserted into the hollow lumen 440 of needle body section 420 to place radial pressure on an inner wall thereof. The pressure from the large stylet is sufficient to break or crack open scored section 421 of needle body section 420 (see FIG. 12C), allowing two or more extending elements such as wires (not shown), one at a time, to be advanced into the epidural or other space, then pushed outside needle body 420 with the stylet. Similar to needle system 5, needle system 405 may include a cutting edge 428 formed on a distal end of needle body section 420 and a removable cap 450 on a proximal end thereof.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A needle system for implanting an extending element, comprising: a needle body configured to be placed in closed state and in open state, wherein an axially extending opening is provided in the needle body in the open state, whereby the extending element, which has been passed through a lumen of the needle body, can be removed from the lumen via the axially extending opening.

2. The needle system of claim 1 wherein the needle body comprises a first body section and a second body section, the axially extending opening being formed in the first body section, the second body section being movable relative to the first body section to place the needle system in the closed state and in the open state.

3. The needle system of claim 2 wherein the second body section is axially slidable relative to the first body section to remove the second body section from connection and into connection with the first body section to place the axially extending opening in the open state or the closed state.

4. The needle system of claim 3 wherein one of the first body section and the second body section includes laterally extending flanges and the other of the first body section and the second body section includes groove positioned to seat the laterally extending flanges.

5. The needle system of claim 3 wherein the second body section is rotatable relative to the first body section to place the axially extending opening in the open state or the closed state.

6. The needle system of claim 1 further comprising a cap removably connectible to a proximal end of the needle body, the cap comprising a passage therethrough which connects with the lumen when the cap is connected to the proximal end of the needle body.

7. The needle system of claim 2 further comprising a cap removably connectible to a proximal end of the needle body, the cap comprising a passage therethrough which connects with the lumen when the cap is connected to the proximal end of the needle body, and wherein the second body section is axially slidable relative to the first body section to remove the second body section from connection with the first body section when the cap is disconnected from the needle body, and the cap secures the second body section in connection with the first body section when the cap is connected to the needle body.

8. The needle system of claim 7 wherein a first connector section is formed on a proximal end of the first body section and a second connector section is formed on a proximal end of the second body section, the first connector section and the second connector section forming a connector when the first body section and the second body section are connected such that the axially extending opening is in the closed state, the cap comprising a cooperating connector to form a removable connection with the connector.

9. The needle system of claim 6 further comprising a stylet configured to be passed through the lumen of the needle body when the needle body is in the open state, wherein passing the stylet through the lumen displaces the extending element to a position from the lumen to a position outside of the needle body.

10. The needle system of claim 6 further comprising one or more movable elements positioned within the lumen of the needle body which are movable to abut and displace the extending element from the lumen to a position outside of the needle body.

11. A method of implanting one or more extending elements, comprising:
inserting a portion of a needle body of a needle system into tissue of a patient while the needle body is in a closed state,
passing the extending element from external to the patient into a lumen of the needle body to extend to a region of interest;
opening an axially extending opening in the needle body to place the needle body in an open state; and
moving a portion of the extending element passing through the lumen from the lumen to outside of the needle body through the axially extending opening.

12. The method of claim 11 wherein the needle body comprises a first body section and a second body section, the axially extending opening being formed in the first body section, the second body section being movable relative to the first body section to place the needle system in the closed state and in the open state.

13. The method of claim 12 wherein moving the second body section relative to the first body section comprises axially sliding the second body section relative to the first body section to remove the second body section from connection with the first body section or to place the second body into connection with the first body.

14. The method of claim 13 wherein one of the first body section and the second body section includes laterally extending flanges and the other of the first body section and the second body section includes grooves positioned to seat the laterally extending flanges.

15. The method of claim 12 wherein moving the second body section relative to the first body section comprises rotating the second body section relative to the first body section.

16. The method of claim 11 wherein a cap is removably connectible to a proximal end of the needle body, the cap comprising a passage therethrough which connects with the lumen when the cap is connected to the proximal end of the needle body.

17. The method of claim 16 wherein the second body section is axially slidable relative to the first body section to remove the second body section from connection with the first body section when the cap is disconnected from the needle body, and the cap secures the second body section in connection with the first body section when the cap is connected to the needle body.

18. The method of claim 16 wherein a first connector section is formed on a proximal end of the first body section and a second connector section is formed on a proximal end of the second body section, the first connector section and the second connector section forming a connector when the first body section and the second body section are connected and the axially extending opening is in a closed state, the cap comprising a cooperating connector to form a removable connection with the connector.

19. The method of claim 11 further comprising passing stylet through the lumen of the needle body when the needle body is in the open state to move the extending element from the lumen via the axially extending opening to a position outside of the needle body.

20. The method of claim 11 further comprising actuating one or more movable elements positioned within the lumen of the needle body and which are movable to abut and move the extending element from the lumen to a position outside of the needle body when the axially extending opening is in the open state.

21. The method of claim 11 further comprising
passing a second extending element from external to the patient into the lumen of the needle body to extend to the region of interest or a second region of interest; and
moving a portion of the second extending element passing through the lumen from the lumen to outside of the needle body through the axially extending opening.
